(19) United States
(12) Patent Application Publication
(10) Pub. No.: US 2010/0198248 A1
(43) Pub. Date: Aug. 5, 2010

(54) SURGICAL DISSECTOR

(75) Inventor: Omar J. Vakharia, Cincinnati, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OH (US)

(21) Appl. No.: 12/364,256

(22) Filed: Feb. 2, 2009

(51) Int. Cl. A61B 17/00; A61B 18/18

(52) U.S. Cl. 606/190; 606/51

(57) ABSTRACT

Various embodiments are directed to a curved-jaw dissector device for use in endoscopic surgical procedures. The device may comprise an end effector, a flexible shaft extending proximally from the end effector; a handle coupled to the proximal portion of the flexible shaft; and a translating member extending from the handle, through the flexible shaft, to the end effector, wherein the translating member is coupled to the handle at an actuator having a first and a second position such that placing the actuator in the first position causes the end effector to be in the closed position and placing the actuator in the second position causes the end effector to be in the open position.

### BACKGROUND

Various embodiments are directed to surgical dissectors for use in minimally invasive surgical procedures.

Minimally invasive procedures are desirable because such procedures can reduce pain and provide relatively quick recovery times as compared to conventional open medical procedures. Many minimally invasive procedures are performed with an endoscope (including without limitation laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. Still less invasive treatments include those that are performed through insertion of an endoscope through a natural body orifice to a treatment region. Examples of this approach include, but are not limited to, cystoscopy, hysteroscopy, esophagogastroduodenoscopy, and colonoscopy.

Many of these procedures employ a flexible endoscope during the procedure. Flexible endoscopes often have a flexible, steerable articulating section near the distal end that can be controlled by the clinician by utilizing controls at the proximal end. Some flexible endoscopes are relatively small (1 mm to 3 mm in diameter), and may have no integral accessory channel (also called biopsy channels or working channels). Other flexible endoscopes, including gastroscopes and colonoscopes, have integral working channels having a diameter of about 2.0 to 3.7 mm for the purpose of introducing and removing medical devices and other accessory devices to perform diagnosis or therapy within the patient. Certain specialized endoscopes are available, such as large working channel endoscopes having a working channel of 5 mm in diameter, which can be used to pass relatively large accessories, or to provide capability to suction large blood clots. Other specialized endoscopes include those having two or more working channels.

### FIGURES

The novel features of the various embodiments are set forth with particularity in the appended claims. The various embodiments, however, both as to organization and methods of operation, together with advantages thereof, may best be understood by reference to the following description, taken in conjunction with the accompanying drawings as follows.

FIG. 1 illustrates one embodiment of an endoscope inserted into the upper gastrointestinal tract of a patient.

FIG. 2 illustrates one embodiment of a distal portion of the endoscope of FIG. 1, which may be used with the surgical dissectors described herein.

FIG. 3 illustrates one embodiment of a surgical dissector, which may be used, with the endoscope of FIG. 1.

FIG. 4 illustrates one embodiment of the end effector of the surgical dissector of FIG. 3.

FIG. 5 illustrates one embodiment of the handle of the surgical dissector of FIG. 3.

FIG. 6 illustrates one embodiment of the handle of FIG. 5 with the handle body not shown.

FIG. 7 illustrates a cross section of one embodiment of the handle of FIG. 5.

FIG. 8 illustrates one embodiment of a slider mechanism from the handle of FIG. 5.

FIG. 9 is an exploded view of the end effector and flexible shaft of one embodiment of the surgical dissector of FIG. 3 having cam-actuated jaws.

FIG. 10 illustrates one embodiment of the surgical dissector of FIG. 3 with a flexible shaft comprising a cut hypotube.

FIGS. 11-14 illustrate one embodiment of the end effector of FIG. 4 transitioning from a closed position shown in FIG. 11 to an open position shown in FIG. 14.

FIG. 15 illustrates one embodiment of the surgical dissector of FIG. 3 having and end effector with a reverse linkage actuation system.

FIG. 16 shows an alternate view of one embodiment of the end effector of FIG. 15 with the clevis not shown.

FIG. 17 illustrates another alternate view of one embodiment of the end effector of FIG. 15 with the near jaw member and link not shown.

FIG. 18 illustrates one embodiment of the end effector of FIG. 15 in an open position.

FIG. 19 is a view of the embodiment shown in FIG. 18 with the near jaw member and link not shown.

FIG. 20 illustrates one embodiment of an end effector where the jaw member comprises a pair of wing features.

FIG. 21 illustrates a more magnified view of one embodiment of the end effector and wing features of FIG. 20.

FIGS. 22-24 show additional views of the end effector and wing features of FIG. 20.

FIGS. 25-26 show one embodiment of an end effector with wing features positioned on both jaw members.

FIG. 27 illustrates one embodiment of the surgical dissector of FIG. 3 for use in electrosurgical applications.

FIG. 28 illustrates one embodiment of an end effector for use in bi-polar electrosurgical applications.

FIG. 29 illustrates one embodiment of an end effector comprising a jaw member with a rounded electrode positioned at the tip of the jaw member.

FIG. 30 illustrates one embodiment of an end effector comprising a jaw member with a hook-shaped electrode.

FIG. 31 illustrates one embodiment of an end effector comprising a jaw member with a wire electrode.

FIG. 32 illustrates another embodiment of an end effector comprising a jaw member with a hook-shaped electrode.

FIG. 33 illustrates one embodiment of an end effector having a jaw member with a strip electrode.

FIG. 34 illustrates one embodiment of an end effector having gauze jaw covers.

### DESCRIPTION

Various embodiments may be directed to surgical dissectors that may be used, for example to dissect tissue during various surgical activities. The surgical dissectors may comprise an end effector having a pair of jaw members that may be transitioned from an open position to a closed position. In some embodiments, the surgical dissectors may be similar to existing “Maryland” dissectors in that the jaw members may curve away from a longitudinal axis of the device. This may make it easier for clinicians to see the distal portion of the jaws around a blood vessel or other viscera while using the dissectors.

The disclosed dissectors may be useful to clinicians for a number of surgical activities. For example, the dissectors may be used to remove an organ, blood vessel, connective tissue or other viscera from the surrounding tissue. The dissector may be inserted through an incision or other cavity between anatomical components while in the closed position. The dissector may then be transitioned to an open position, which may cause the anatomical components to be separated from one another. For example, the dissectors may be used to remove the gall bladder from the liver bed. In some embodiments, the inner surfaces of the jaws of the dissector may have teeth, allowing the clinician to grip and/or tear tissue. Also, various embodiments may include one or more electrodes positioned on the jaws, making them suitable for use in electrosurgical applications.

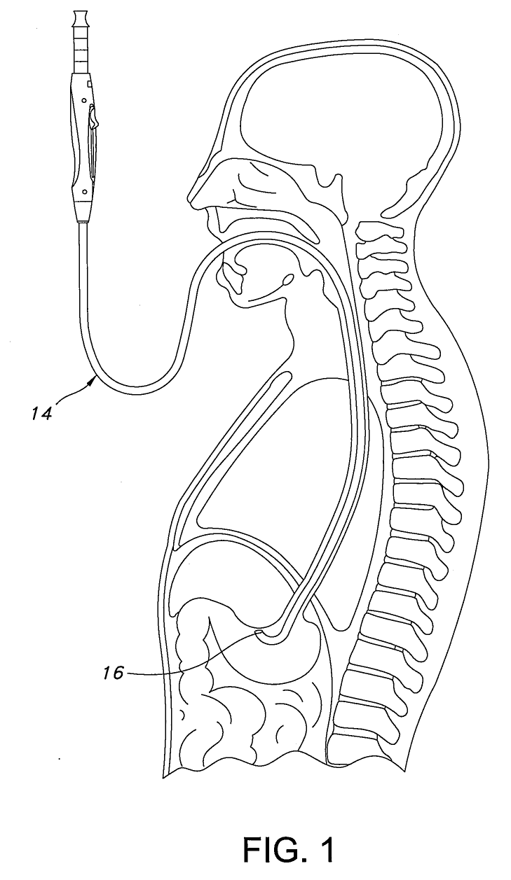

FIG. 1 illustrates one embodiment of an endoscope 14 (illustrated here as a gastroscope) inserted into the upper gastrointestinal tract of a patient. The endoscope 14 has a distal end 16 that may include various optical channels, illumination channels, and working channels. According to various embodiments, the endoscope 14 may be a flexible endoscope, and may be introduced via natural orifices.

In one embodiment, Natural Orifice Translumenal Endoscopic Surgery (NOTES)™ techniques may be employed to introduce the endoscope 14 and various instruments into the patient and carry out the various procedures described herein. A NOTES™ technique is a minimally invasive therapeutic procedure that may be employed to treat diseased tissue or perform other therapeutic operations through a natural opening of the patient without making incisions in the abdomen. A natural opening may be the mouth, anus, and/or vagina. Medical implantable instruments may be introduced into the patient to the target area via the natural opening. In a NOTES™ technique, a clinician inserts a flexible endoscope into one or more natural openings of the patient to view the target area, for example, using a camera. During endoscopic surgery, the clinician inserts surgical devices through one or more lumens or working channels of the endoscope 14 to perform various key surgical activities (KSA). These KSAs include forming an anastomosis between organs, performing dissections, repairing ulcers and other wounds. Although the devices and methods described herein may be used with NOTES™ techniques, it will be appreciated that they may also be used with other surgical techniques including, for example, other endoscopic techniques, and laparoscopic techniques.

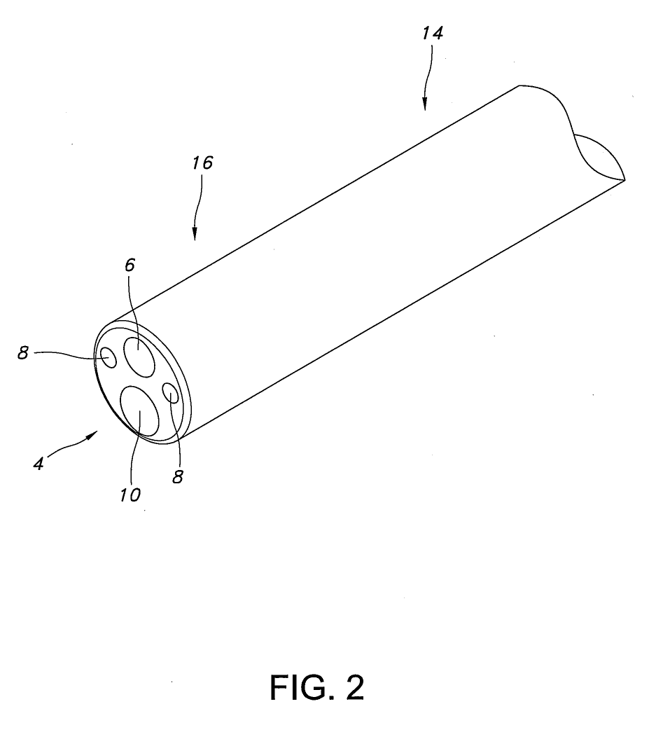

FIG. 2 illustrates one embodiment of a distal portion 16 of the endoscope 14, which may be used with the surgical dissectors described herein. The example endoscope 14 shown comprises a distal face 4, which defines the distal ends of illumination channels 8, an optical channel 6 and a working channel 10. The illumination channels 8 may comprise one or more optical fibers or other suitable waveguides for directing light from a proximally positioned light source (not shown) to the surgical site. The optical channel 6 may comprise one or more optical fibers or other suitable waveguides for receiving and transmitting an image of the surgical site proximally to a position where the image may be viewed by the clinician operating the endoscope 14. As described above, the working channel 10 may allow the clinician to introduce one or more surgical tools to the surgical site. Examples of such surgical tools include scissors, cautery knives, suturing devices, and dissectors. It will be appreciated that the endoscope 14 is but one example of an endoscope that may be used in accordance with various embodiments. Endoscopes having alternate configurations of optical channels 6, illumination channels 8 and/or working channels 10 may also be used.

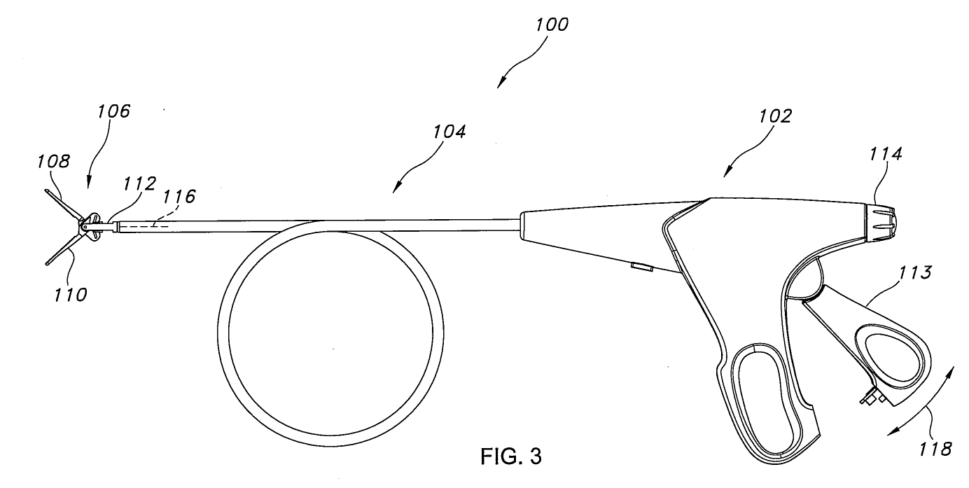

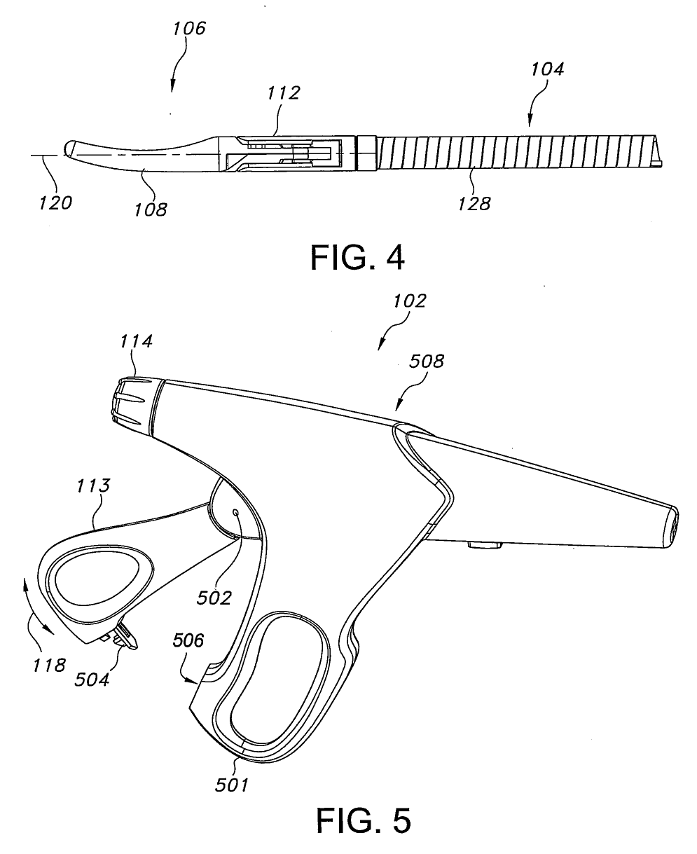

FIG. 3 illustrates one embodiment of a surgical dissector 100, which may be used, for example, with an endoscope such as the endoscope 14. The dissector 100 may comprise a handle assembly 102, a flexible shaft 104 and an end effector 106. The end effector 106 may comprise a first jaw member 108 and a second jaw member 110. The first jaw member 108 and second jaw member 110 may be connected to a clevis 112, which, in turn, may be coupled to the flexible shaft 104. FIG. 4 illustrates one embodiment of the end effector 106 of the surgical dissector 100. As illustrated, the first jaw member 108 and second jaw member (obscured by jaw member 108 in FIG. 4) are curved relative to an axis 120 of the end effector 106.

Referring back to FIG. 3, a translating member 116 may extend within the flexible shaft 104 from the end effector 106 to the handle 102. The translating member 116 may be made from any suitable material. For example, the translating member may be, a metal wire (e.g., a tri-layered steel cable), a plastic or metal shaft. At the handle 102, the flexible shaft 104 may be directly or indirectly coupled to an actuator 113. In use, a clinician may cause the actuator 113 to pivot along arrow 118 from a first position to a second position. When the actuator moves from the first position to the second position, it may translate the translating member 116 distally or proximally. Distal or proximal motion of the translating member 116 may, in turn, cause the end effector 106 to transition from an open position to a closed position.

FIG. 5 illustrates one embodiment of the handle 102 of the surgical dissector 100. The actuator 113 may pivot about pivot point 502 along arrow 118 as shown. The pivot point 502 may represent a pin or other connector fastening the actuator to the handle body 508. The handle body 508 may define a grip 501 opposite the actuator 113 as shown. In one example, use, the clinician may place one or more fingers through the grip 501, allowing the clinician to manipulate the actuator 113 with a thumb. According to various embodiments, the actuator 113 may comprise a lock element 504 configured to be securely received into a lock cavity 506. The lock element 504 and cavity 506 may allow the clinician to secure the actuator 113, and thus the end effector 106, into a given position.

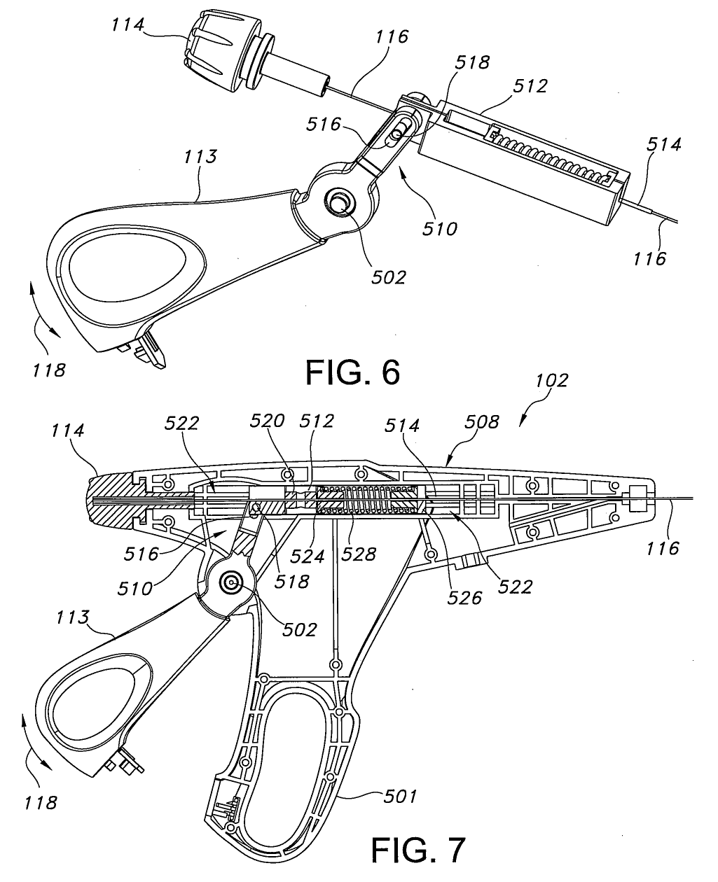

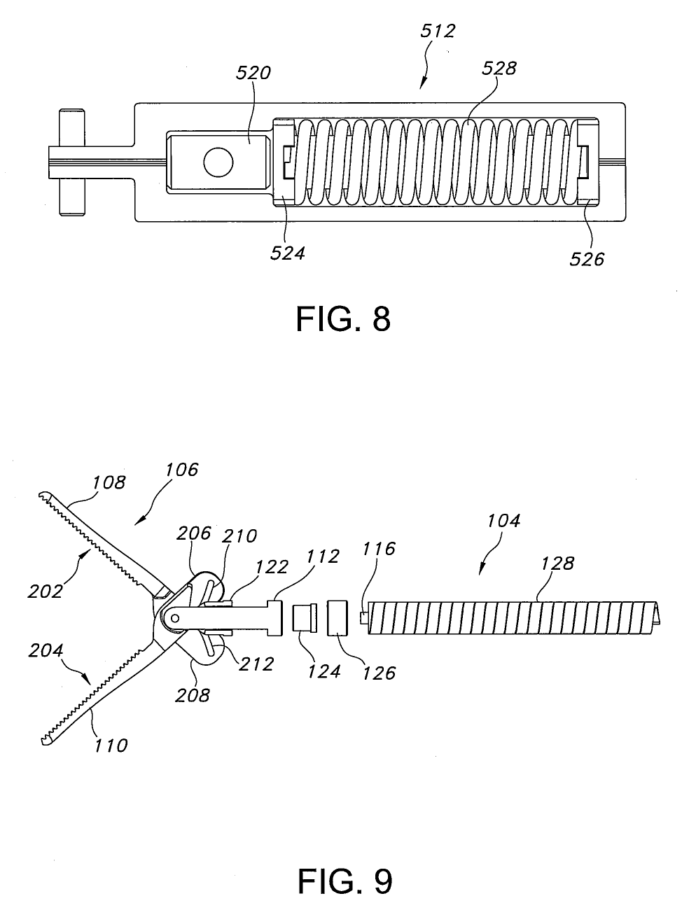

FIG. 6 illustrates one embodiment of the handle 102 with the handle body 508 not shown. The actuator 113 is shown with a pair of arms 510 defining slots 516. The arms 510 receive a pin 518 to slidably couple the actuator to a slider mechanism 512. FIG. 7 illustrates a cross section of one embodiment of the handle 102. FIG. 8 illustrates one embodiment of the slider mechanism 512. The translating member 116 is received at the distal portion of the handle body 508 and extends proximally to the slider mechanism 512. Within the slider mechanism 512, the translating member 116 may be received by a pair of spring holders 524, 526 and a collar 520. From the collar 528, the translating member 116 may extend proximally to the rotation knob 114. The translating member 116 may be securely fastened to the collar 520 such that the translating member 116 cannot translate distally and proximally with respect to the collar 520.

In use, the clinician may move the actuator 113 towards the grip 501 to force the translating member 116 proximally. The resulting rotation of the actuator 113 about the pivot point 502 may pull the slider mechanism 512 proximally within the cavity 522 defined by the handle body 508. This may also pull the collar 520 and translating member 116 proximally. Spring 528 may resist motion of the slider mechanism 512 and thus the translating member 116. To move the translating member 116 distally, the clinician may pivot the actuator 113 away from the grip 501 about the pivot point 502. This may force the slider mechanism 512 and thus the translating member 116 distally. A translating member sleeve 514 may be provided between the distal portion of the slider mechanism 512 and the distal tip of the handle 102. The sleeve 514 may serve to prevent buckling of the translating member 116 when it is forced distally.

FIG. 9 illustrates an exploded view of the end effector 106 and flexible shaft 104 of one embodiment of the surgical dissector 100 having cam-actuated jaws. The jaw members 108, 110 may each comprise inner surfaces 202, 204. When the end effector 106 is in the closed position, the inner surfaces 202, 204 may be in contact with one another. In the embodiment illustrated in FIG. 9, the inner surfaces 202, 204 comprise a plurality of teeth configured to interlock with one another when the end effector 106 is in the closed position. The jaw members 108, 110 may also comprise proximal cam members 206, 208. Each of the cam members 206, 208 may define a cam slot 210, 212. A shuttle 122 may comprise one or more pin features 214 (not shown in FIG. 9) that ride in the cam slots 210, 212. For example, the shuttle 122 may comprise a single pin feature 214 extending through both sides or separate pin features 214 on each side. In use, the shuttle 122 may be coupled to the translating member 116. Distal motion of the translating member 116 may cause corresponding distal motion of the shuttle 122, which may, in turn, force the pin features 214 to slide within the cam slots 210, 212, forcing the jaw members 108, 110 into an open position.

According to various embodiments, the end effector 106 may be rotatably coupled to the flexible shaft 104. For example, an outer coupler 126 may be fastened to the flexible shaft 104. An inner coupler 124 may be fastened within the outer coupler 126 such that the inner coupler 124 can rotate relative to the outer coupler 126 and the flexible shaft 104. The inner coupler 124 may also be coupled to the clevis 112 (and hence the end effector 106). Accordingly, the end effector 106 may be rotatable, with the inner coupler 124, about the outer coupler 126 and the flexible shaft 104. As described above, the translating member 116 may be coupled to the end effector 106, for example, via the shuttle 122. The clinician may bring about rotation of the end effector 106 by rotating the translating member 116. For example, referring to FIG. 5-7, the handle 102 may comprise a knob 114 or other control device allowing the clinician to rotate the translating member 116.

The flexible shaft 104 may be made from any suitable material and/or device. In various embodiments the flexible shaft 104 may be made from a material or device that is flexible and also able to withstand tension and compression forces to avoid significant losses in the opening and closing forces provided by the clinician via the actuator 113. For example, when the actuator 118 causes the translating member 116 to move distally, the flexible shaft 104 may be placed in compression. When the actuator 118 causes the translating member 116 to move proximally, the flexible shaft 104 may be placed in tension. Excessive compression or stretching of the flexible shaft 104 may attenuate the force ultimately provided to open or close the end effector 106.

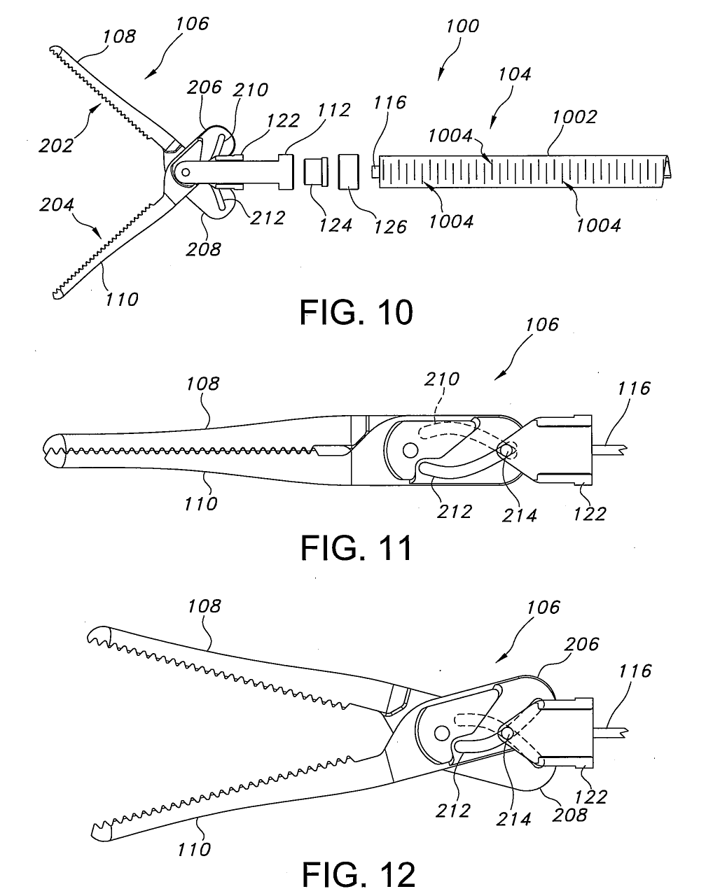

In various embodiments, the flexible shaft may comprise a coil pipe 128, as illustrated in FIGS. 4 and 9. The coil pipe 128 may be made from wire or a narrow ribbon of material formed into a cylindrical coil. The coiled nature of the coil pipe 128 may cause it to perform well in compression. In tension, however, the coil pipe 128 may tend to expand, thus attenuating the force applied to the end effector 106. The attenuation may be minimized by selecting a coil pipe 128 with a high pre-load. This may make the coil pipe 128 relatively stiff and more difficult to bend, but may also improve its performance in tension. FIG. 10 illustrates another embodiment of the surgical dissector 100 with a flexible shaft 104 comprising a cut hypotube 1002 in place of the coil pipe 128. The cut hypotube 1002 may be a cylindrical piece of material (e.g., surgical steel or other metal) with a plurality of cuts or cut-out features 1004. The cuts may allow the hypotube 1002 to bend. Because the hypotube 1002 may bend on the cuts, the spatial frequency of the cuts in any given portion of the hypotube 1002 may determine the flexibility of that portion. A higher spatial frequency of cuts may correspond to a higher flexibility. Because the hypotube 1002 is not configured to stretch under ordinary operating conditions, it may provide increased tensile performance compared to the coil pipe 128.

FIGS. 11-14 illustrate one embodiment of the end effector 106 transitioning from a closed position shown in FIG. 11 to an open position shown in FIG. 14. Referring to FIG. 11, the end effector 106 is shown in the closed position. The jaw members 108, 110 are illustrated in contact with one another. The shuttle 122 is shown coupled to the translating member 116 and in a proximal position. For example, a clinician operating the actuator 113 may have caused the translating member 116 to translate through the flexible shaft 104 in a proximal direction. This may, in turn, have caused the shuttle 122 to assume the proximal position shown. When the shuttle 122 is in the proximal position the pins 214 may be positioned within the slots 210, 212 such that the jaw members 108, 110 are in the closed position.

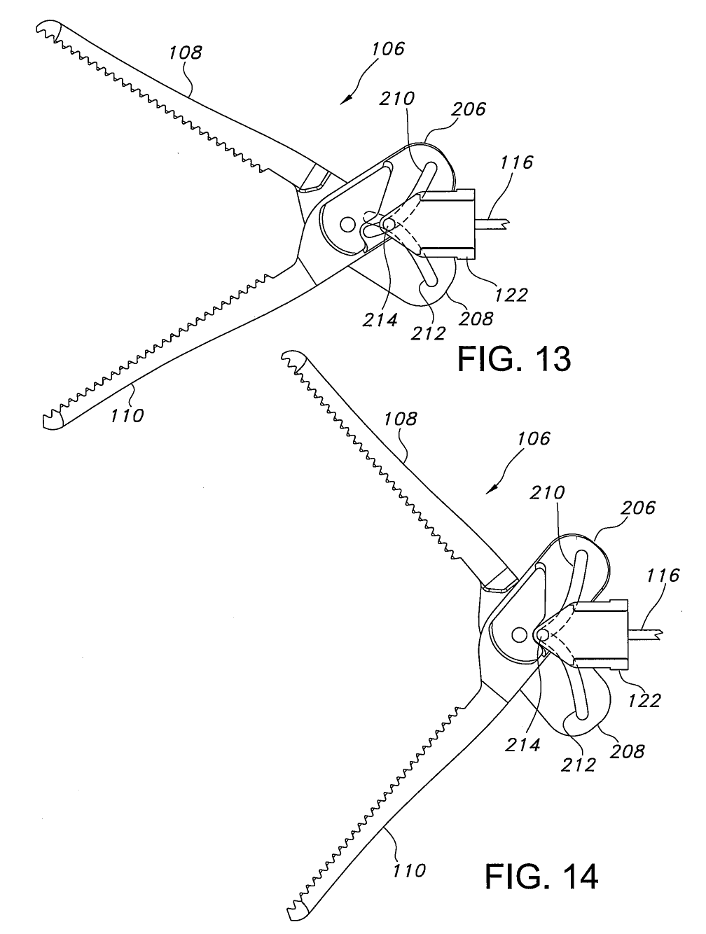

FIGS. 12 and 13 illustrate one embodiment of the end effector 106 transitioning from the closed position to the open position. As the translating member 116 and shuttle 122 are pushed distally, the pins 214 may also move distally within the cam slots 210, 212. Due to the curvature of the cam slots 210, 212, this may force the jaw members 108, 110 into the open position. In FIG. 14, the end effector 106 is shown with the shuttle 122 in its fully distal position and the jaw members 108, 110 in their fully open position. As shown in FIG. 14, the jaw members 108, 110 form a fully open aperture angle of about 90°. It will be appreciated, however, that various embodiments may have different fully open aperture angles. For example, dissectors with aperture angles of 40° may be used. Also, dissectors with aperture angles of 180° may be used.

It will be appreciated that the profile (e.g., shape) of the cam slots 210, 212, may bring about a mechanical advantage, lessening the force necessary to open or close the end effectors 106. For example, configuring the cam slots 210, 212 with a shallow profile may reduce the mechanical advantage between the actuator 113 and the end effector 106. This may, in turn, minimize the movement of the actuator 113 that is necessary to open the end effector 106, but maximize the required force. Similarly, configuring the cam slots 210, 212 with a more curved profile may increase the mechanical advantage between the actuator 113 and the end effector 106. This may decrease the force that the clinician must apply to the actuator 113, but increase the necessary movement.

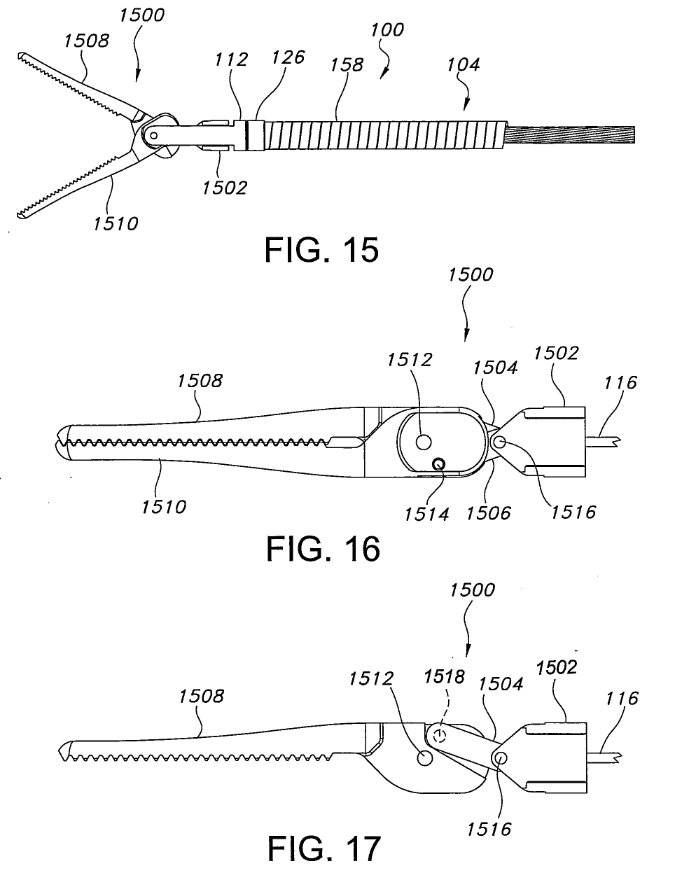

FIG. 15 illustrates one embodiment of the surgical dissector 100 having a reverse linkage actuation system. The end effector 1500 may comprise jaw members 1508 and 1510 as well as a shuttle 1502. Links 1504, 1506 (not shown in FIG. 15) may couple the shuttle 1502 to the jaw members 1508, 1510. Although FIG. 15 illustrates an embodiment where the flexible shaft 104 comprises a coil pipe 158, it will be appreciated that a cut hypotube may be substituted in various embodiments. FIG. 16 shows an alternate view of one embodiment of the end effector 1500 with the clevis 112 not shown. The jaw members 1508, 1510 may pivot from the open to the closed position about pivot point 1512. The links 1504, 1506 are fastened to the shuttle 102 at pivot point 1516. The link 1506 is also coupled to the jaw member 1510 at pivot point 1514. Although not shown in FIG. 16, the link 1504 may be coupled to the jaw member 1508 at a pivot point 1518 similar to the pivot point 1514. FIG. 17 illustrates another alternate view of one embodiment of the end effector 1500 with the jaw member 1510 and the link 1506 not shown. The pivot point 1518 is visible along with the link 1504.

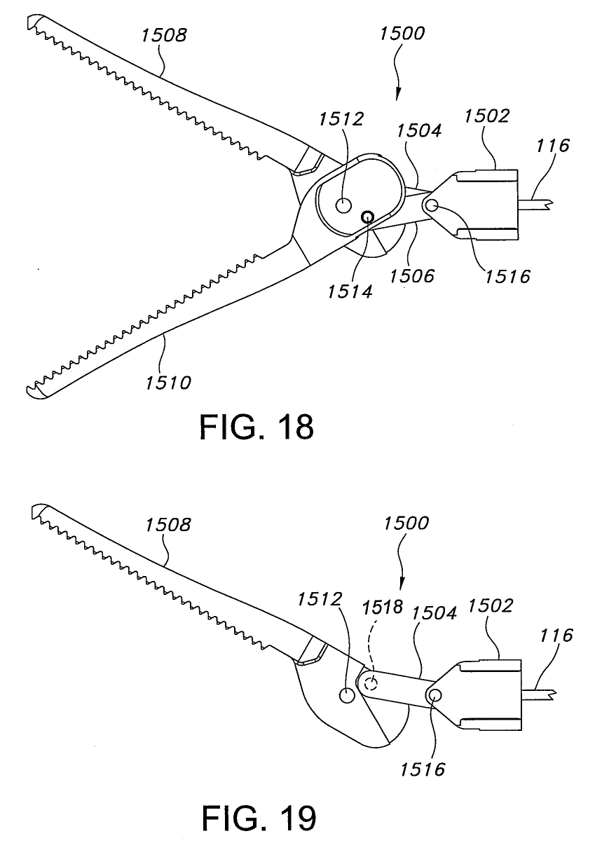

According to various embodiments, the pivot points 1514 and 1518 may be positioned on the respective jaw members relative to pivot point 1512 such that distal movement of the shuttle 1502 causes the jaw members 1508, 1510 to close. FIG. 18 illustrates one embodiment of the end effector 1500 in an open position. As shown in FIG. 18, the shuttle 1502 is in a more proximal position than that shown in FIGS. 15 and 16. As a result, the links 1504, 1506 are pulled to a more proximal position causing the jaw members 1508, 1510 to pivot about the pivot point 1512 to the open position shown. FIG. 19 is a view of the embodiment shown in FIG. 18 with the jaw member 1510 and link 1506 not shown.

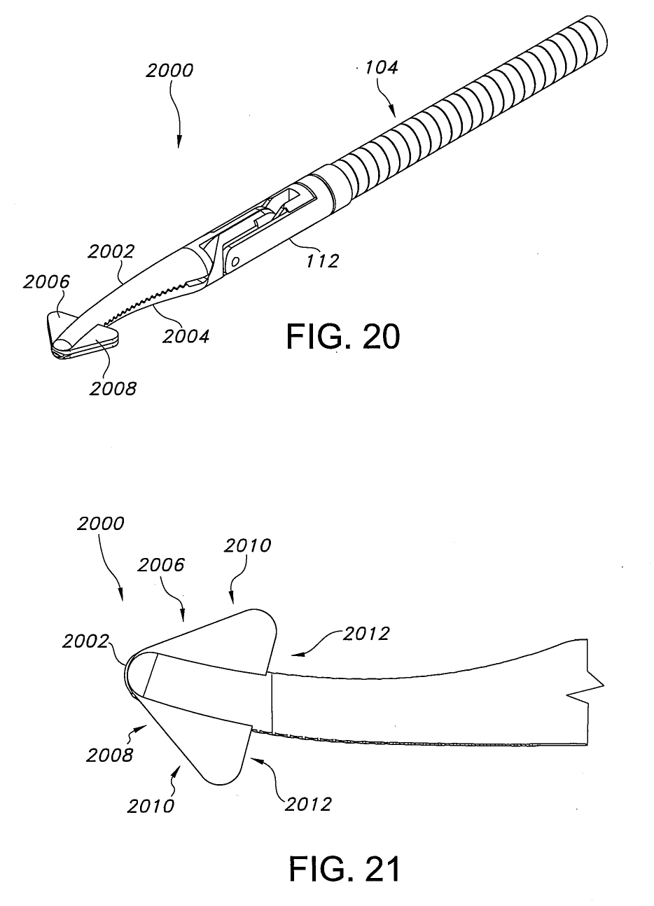

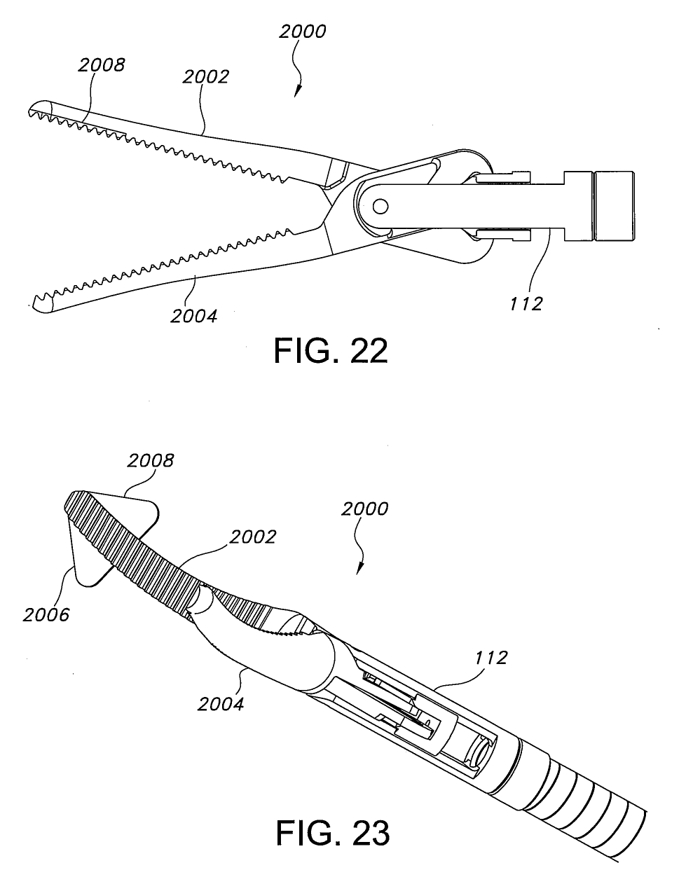

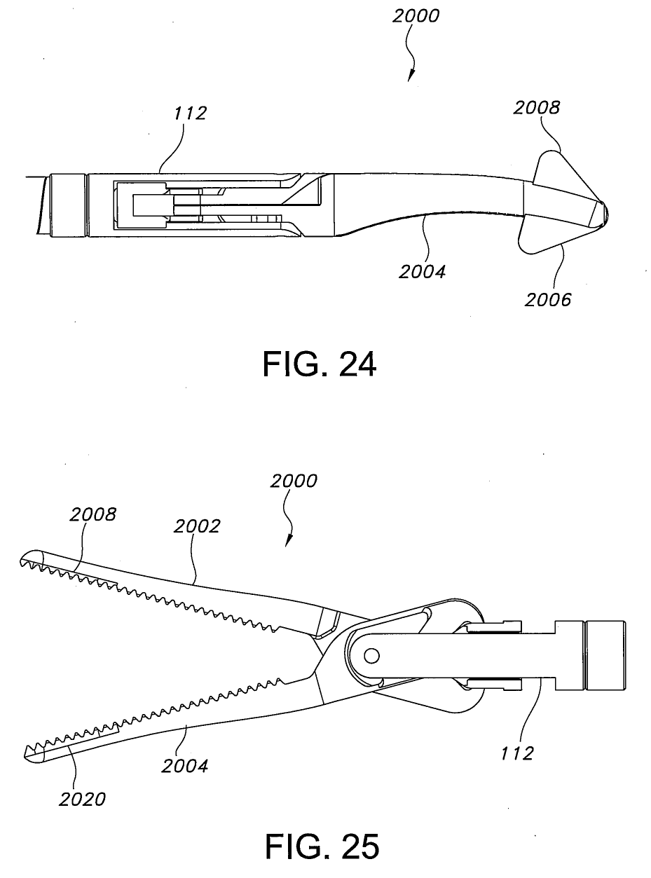

FIG. 20 illustrates one embodiment of the end effector 2000 where the jaw member 2002 comprises a pair of wing features 2006, 2008. The wing features 2006, 2008 extend away from a longitudinal axis of the jaw member 2002. FIG. 21 illustrates a more magnified view of one embodiment of the end effector 2000 and wing features 2006, 2008. FIGS. 22-24 show additional views of the end effector 2000 and wing features 2006, 2008. The wing features 2006, 2008 may be made from any suitable material including, for example, surgical steel or plastic. According to various embodiments, the wing features 2006, 2008 may be delta shaped. For example, proximally positioned portions of the wing features 2006, 2008 may extend farther from the jaw member 2002 than distally positioned portions of the wing features 2006, 2008. In various embodiments, the wing features 2006, 2008 may define distally facing leading edges 2010 and proximally-facing trailing edges 2012. The leading edges 2010 may be sharpened to a point. The trailing edges 2012 may be sharpened, or may be blunt.

The wing features 2006, 2008 may be useful in dissections and other surgical activities. For example, the leading edges 2010 of the wing features 2006, 2008 may serve to spread tissue. In various surgical uses, the end effector 2000 may be slid between tissue components (e.g., a gall bladder and a liver bed). The leading edges 2010 of the wing features 2006, 2008 may serve to sever some of the intermediate and connective tissue joining the tissue components. Once the end effector 2000 is in place relative to tissue, the trailing edges 2012 may serve as an anchor to prevent tissue from sliding off of the distal portions of the jaw member 2002, for example, while the end effector 2000 is transitioning to the open position.

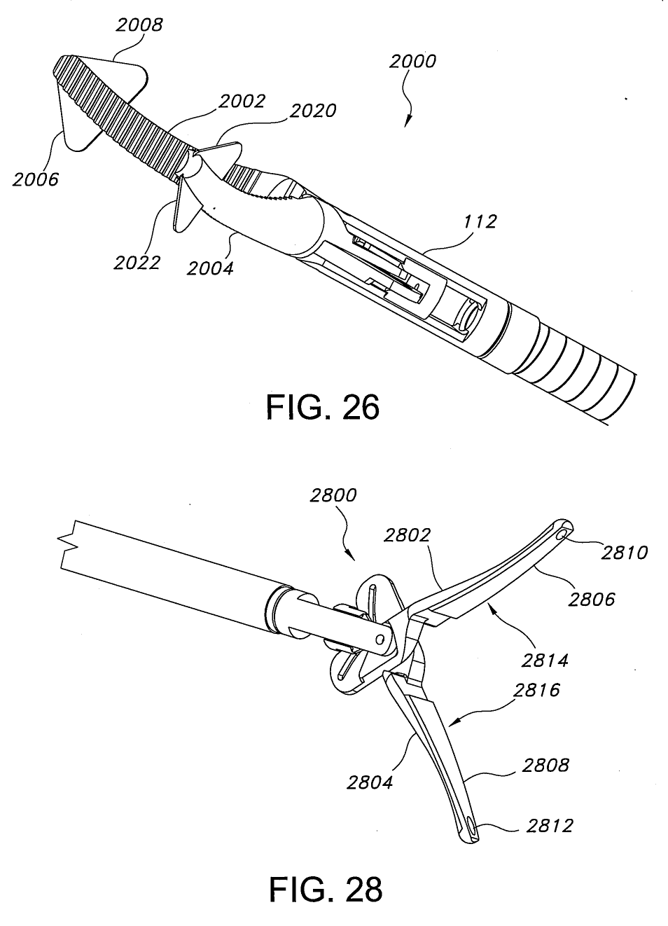

In FIGS. 20-24, the wing features 2006, 2008 are shown on the jaw member 2002 only. It will be appreciated, however, that the jaw member 2004, or both jaw members 2002, 2004 may have wing features. For example, FIGS. 25-26 show one embodiment of the end effector 2000 with wing features 2020, 2022 positioned on the jaw member 2004 in addition to the wing features 2006, 2008 positioned on the jaw member 2002.

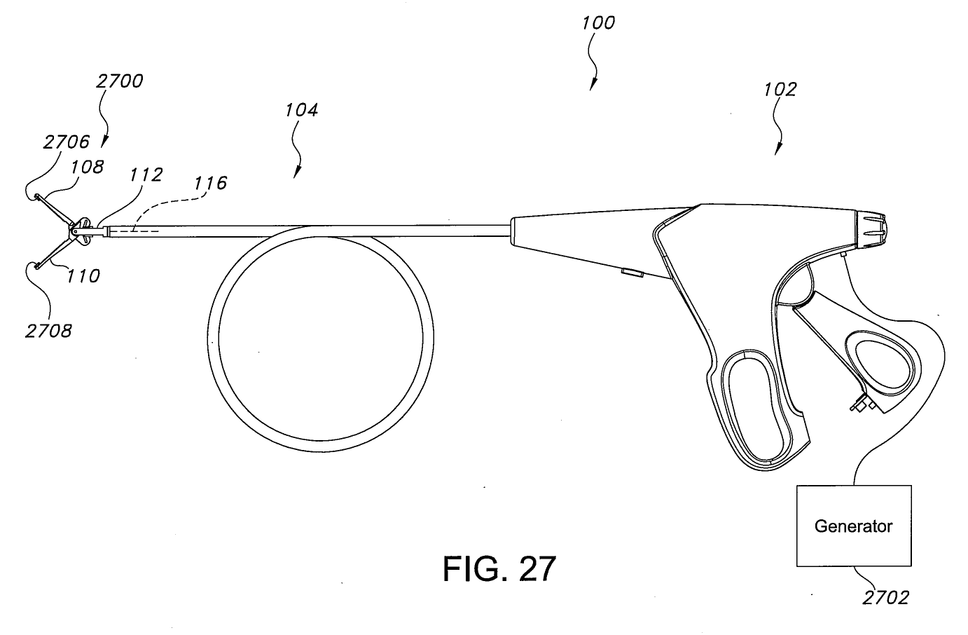

According to various embodiments, some or all of the jaw members 108, 110 may include, or serve as electrodes in monopolar or bi-polar electrosurgical applications including, for example, cutting and coagulation. FIG. 27 illustrates one embodiment of the surgical dissector 100 for use in electrosurgical applications. The jaw members 2706, 2708 of the end effector 2700 may comprise respective electrodes 2706, 2708. The electrodes may be connected to an electrosurgical generator 2702 via wires (not shown) extending from the end effector 2700 through the flexible shaft 104 and handle 102. The generator 2702 may generate any suitable type of signal for electrosurgical applications. For example, the generator 2702 may make various alternating current (A/C) and/or direct current (D/C) signals are suitable voltages, currents and, for A/C currents, at suitable frequencies and wave patterns. According to various embodiments, the surgical dissector 100 may be configured for monopolar operation. In this case, the end effector 2700 may comprise a single electrode, rather than two. According to various embodiments, all or a portion of the end effector 2700 may serve as the single electrode. It will be appreciated that all of the electrode configurations described below may be used with any of the features described above including, for example, cam actuation, reverse linkage actuation, wing features, etc.

FIG. 28 illustrates one embodiment of an end effector 2800 for use in bi-polar electrosurgical applications. The end effector 2800 may comprise a pair of jaw members 2802, 2804 that may operate in a manner similar to those of the end effector 106 described above. Referring first to jaw member 2802, its inner surface 2814 may comprise an insulating member 2806 and an electrode 2810. The insulating member 2806 may serve to electrically isolate the electrode 2810 from the remainder of the jaw member 2802. Jaw member 2804 may have a similar insulating member 2808 and electrode 2812. The insulating members 2806, 2808 may be made from any suitable electrically insulating material including, for example, plastic. The electrodes 2810, 2812 may be made from any suitable electrically conducting material including, for example, surgical steel or another metal.

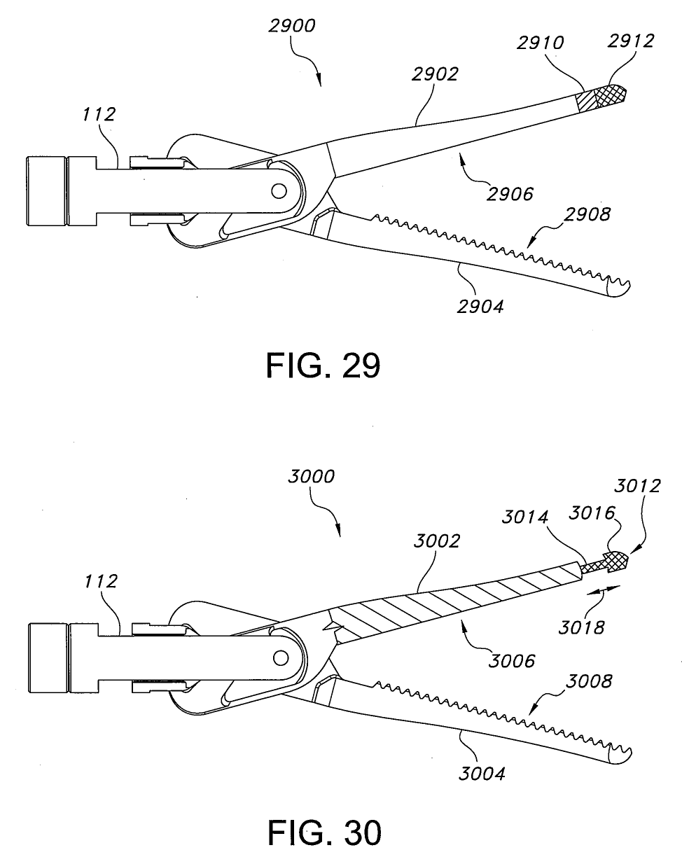

FIG. 29 illustrates one embodiment of an end effector 2900 comprising a jaw member 2902 with a rounded electrode 2912 positioned at the tip of the jaw member 2902. The electrode 2912 may be electrically isolated from the remainder of the jaw member 2902 by an insulating member 2910. An inner surface 2906 of the jaw member 2902 may be smooth, as shown, or may define teeth or other gripping features. In FIG. 29, the opposite jaw member 2904 is shown without an electrode and with an inner surface 2908 defining a plurality of teeth. It will be appreciated, however, that in various embodiments, the inner surface 2908 of the jaw member 2904 may be smooth or may comprise various other gripping features. Also, according to various bi-polar embodiments, the jaw member 2904 may comprise an electrode, which may be similar to the electrode 2912.

FIG. 30 illustrates one embodiment of an end effector 3000 comprising a jaw member 3002 with a hook-shaped electrode 3012. The electrode 3012 may be positioned at the distal tip of the jaw member 3002 and may comprise a shaft portion 3014 and a hook portion 3016. The hook portion 3016 of the electrode 3012 may be proximally directed and may facilitate cutting and coagulating activities. According to various embodiments, the electrode 3012 may be slidable coupled to the jaw member 3002 such that the electrode 3012 is translatable distally and proximally in the direction of arrow 3018. This may give the clinician additional control over the position of the electrode 3012 when it is activated. A clinician may be able to move the electrode 3012 distally and proximally by pulling the wire (not shown) connecting the electrode 3012 to the generator 2702 distally and proximally. According to various embodiments, the handle 102 may comprise a suitable control for allowing the clinician to move the wire distally and proximally. Although the end effector 3000 is shown in a monopolar configuration, it will be appreciated that, in various embodiments, the jaw member 3004 may also comprise an electrode (not shown). Also, the inner surfaces 3006, 3008 of the jaw members 3002, 3004 may be smooth or may comprise teeth or other gripping features.

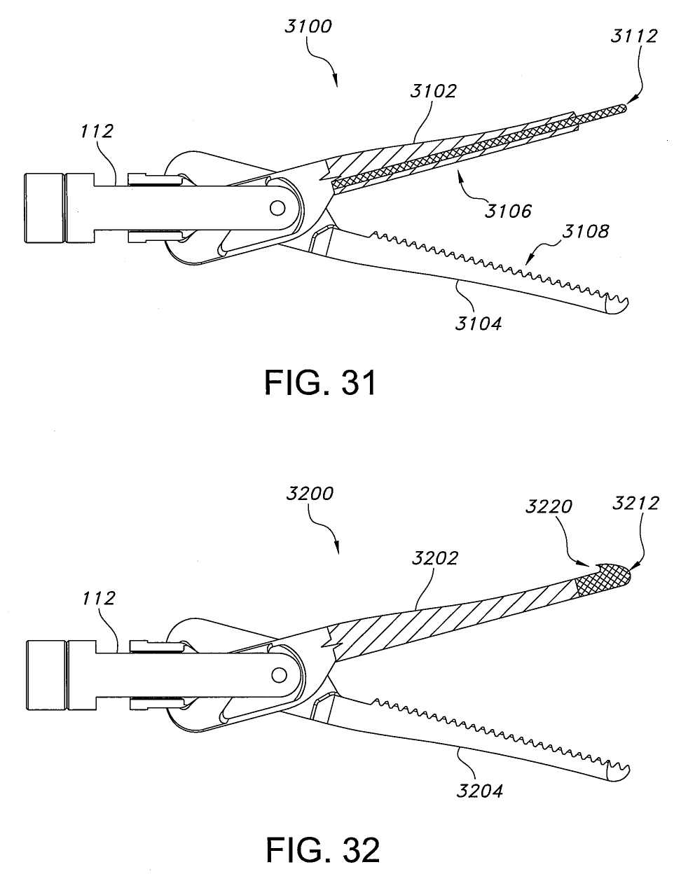

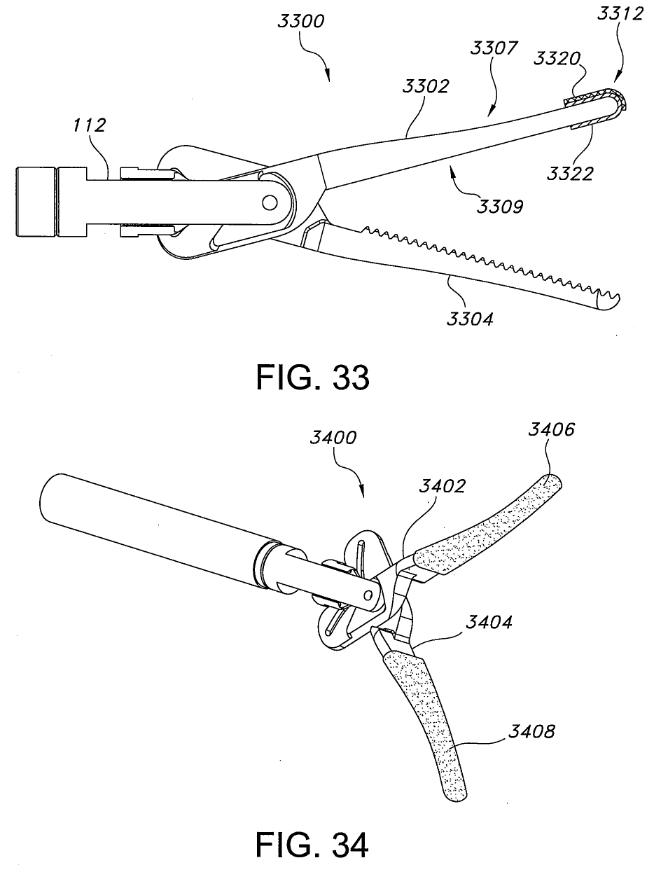

FIG. 31 illustrates one embodiment of an end effector 3100 comprising a jaw member 3102 with a wire electrode 3112. The jaw member 3102 is shown in cross-section illustrating the wire electrode 3112 extending through the jaw member 3102. The wire electrode 3112 may extend proximally through the flexible shaft 104 and handle 102 to the generator 2702. According to various embodiments, wire electrode 3112 may be movable distally and proximally, for example, as described above. FIG. 32 illustrates another embodiment of an end effector 3200 comprising a jaw member 3202 having a hook-shaped electrode 3212. The electrode 3212 may comprise a proximally-directed hook feature 3220 that may be used when cutting and/or cauterizing tissue. FIG. 33 illustrates one embodiment of an end effector 3300 having a jaw member 3302 with a strip electrode 3312. The strip electrode 3312 may comprise an electrically conducting member 3320, which may be in electrical communication with the generator 2702. The strip electrode 3312 may also comprise an electrically insulating member 3322, which may electrically isolate the conducting member 3320 from the remainder of the jaw member 3302. All or a portion of the strip electrode 3312 may be positioned on an outer surface 3307 opposite the inner surface 3309 of the jaw member 3302. It will be appreciated that the various end effectors 3100, 3200 and 3300 may be embodied with monopolar electrodes, as shown, or, in various embodiments, may include additional electrodes (e.g., on jaw members 3104, 3204, 3304). Also, the respective inner surfaces of the jaw members may be smooth or may have teeth or other suitable gripping features.

FIG. 34 illustrates one embodiment of an end effector 3400 having gauze jaw covers. The end effector 3400 may comprise jaw members 3402, 3404 as described above. Each jaw member 3402, 3404 may comprise a respective jaw cover 3406, 3408. The jaw covers 3406, 3408 may be made from a gauze material which may serve to increase friction between the jaw members 3403, 3404 and surrounding tissue and may also serve to soak up blood and other fluids that may be present at the surgical site, thus improving the view of the clinician.

In various embodiments, surgical instruments utilizing various embodiments of the surgical dissector 100, with the various end effectors and actuating mechanisms described herein may be employed in conjunction with a flexible endoscope, such as a GIF-100 model available from Olympus Corporation, for example. In at least one such embodiment, the endoscope, a laparoscope, or a thoracoscope, for example, may be introduced into the patient trans-anally through the colon, the abdomen via an incision or keyhole and a trocar, or trans-orally through the esophagus or trans-vaginally through the cervix, for example. These devices may assist the clinician to guide and position the surgical dissector 100 near the tissue treatment region to treat diseased tissue on organs such as the liver, for example. In another embodiment, these devices may be positioned to treat diseased tissue near the gastrointestinal (GI) tract, esophagus, and/or lung, for example. In various embodiments, the endoscope may comprise a flexible shaft where the distal end of the flexible shaft may comprise a light source, a viewing port, and at least one working channel. In at least one such embodiment, the viewing port may transmit an image within its field of view to an optical device such as a charge coupled device (CCD) camera within the endoscope, for example, so that an operator may view the image on a display monitor (not shown).

It will be appreciated that the terms “proximal” and “distal” are used herein with reference to a clinician manipulating an end of an instrument extending from the clinician to a surgical site (e.g., through a trocar, through a natural orifice or through an open surgical site). The term “proximal” refers to the portion closest to the clinician, and the term “distal” refers to the portion located away from the clinician. It will be further appreciated that for conciseness and clarity, spatial terms such as “vertical,” “horizontal,” “up,” and “down” may be used herein with respect to the drawings. However, surgical instruments are used in many orientations and positions, and these terms are not intended to be limiting and absolute.

While several embodiments have been illustrated and described, and while several illustrative embodiments have been described in considerable detail, the described embodiments are not intended to restrict or in any way limit the scope of the appended claims to such detail. Additional advantages and modifications may readily appear to those skilled in the art. Those of ordinary skill in the art will readily appreciate the different advantages provided by these various embodiments.

While several embodiments have been described, it should be apparent, however, that various modifications, alterations and adaptations to those embodiments may occur to persons skilled in the art with the attainment of some or all of the advantages of the embodiments. For example, according to various embodiments, a single component may be replaced by multiple components, and multiple components may be replaced by a single component, to perform a given function or functions. The described embodiments are therefore intended to cover all such modifications, alterations and adaptations without departing from the scope of the appended claims.

The devices disclosed herein may be designed to be disposed of after a single use, or they may be designed to be used multiple times. In either case, however, the device may be reconditioned for reuse after at least one use. Reconditioning may include a combination of the steps of disassembly of the device, followed by cleaning or replacement of particular pieces, and subsequent reassembly. In particular, the device may be disassembled, and any number of particular pieces or parts of the device may be selectively replaced or removed in any combination. Upon cleaning and/or replacement of particular parts, the device may be reassembled for subsequent use either at a reconditioning facility, or by a surgical team immediately prior to a surgical procedure. Those of ordinary skill in the art will appreciate that the reconditioning of a device may utilize a variety of different techniques for disassembly, cleaning/replacement, and reassembly. Use of such techniques, and the resulting reconditioned device, are all within the scope of this application.

Preferably, the embodiments described herein will be processed before surgery. First a new or used instrument is obtained and, if necessary, cleaned. The instrument may then be sterilized. In one sterilization technique, the instrument is placed in a closed and sealed container, such as a plastic or TYVEK® bag. The container and instrument are then placed in a field of radiation that may penetrate the container, such as gamma radiation, x-rays, or higher energy electrons. The radiation kills bacteria on the instrument and in the container. The sterilized instrument may then be stored in the sterile container. The sealed container keeps the instrument sterile until it is opened in the medical facility.

Any patent, publication, or other disclosure material, in whole or in part, that is said to be incorporated by reference herein is incorporated herein only to the extent that the incorporated materials do not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The embodiments are not to be construed as limited to the particular embodiments disclosed. The embodiments are therefore to be regarded as illustrative rather than restrictive. Variations and changes may be made by others without departing from the scope of the claims. Accordingly, it is expressly intended that all such equivalents, variations and changes that fall within the scope of the claims be embraced thereby.

In summary, numerous benefits have been described which result from employing the embodiments described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical applications to thereby enable one of ordinary skill in the art to utilize the various embodiments and with various modifications as are suited to the particular use contemplated. It is intended that the claims submitted herewith define the overall scope.

What is claimed is:

1. A curved-jaw dissector device for use in endoscopic surgical procedures, the device comprising:
an end effector comprising:
a first jaw member defining a first surface; and
a second jaw member defining a second surface, wherein the first and second jaw members have a common pivot point such that the end effector has an open position where the first and second surfaces are pivoted away from one another and a closed position where the first and second surfaces are pivoted towards each other, and wherein the first jaw member and the second jaw member are curved away from a longitudinal axis of the end effector;
a flexible shaft extending proximally from the end effector;
a handle coupled to a proximal portion of the flexible shaft;
a translating member extending from the handle, through the flexible shaft, to the end effector, wherein the translating member is coupled to the handle at an actuator having a first and a second position such that placing the actuator in the first position causes the end effector to be in the closed position and placing the actuator in the second position causes the end effector to be in the open position;
wherein the first jaw member comprises:
a first wing feature extending away from a longitudinal axis of the first jaw member, wherein a narrow end of the first wing feature is pointed distally; and
a second wing feature extending away from the longitudinal axis of the first jaw member and opposite the first wing feature, wherein a narrow end of the second wing feature is pointed distally.

2. The device of claim 1, wherein leading edges of the first and second wing features are sharpened.

3. The device of claim 1, wherein the first and second jaw members comprise a material selected from the group consisting of surgical steel and plastic.

4. The device of claim 1, wherein the first and second wing features comprise a material selected from the group consisting of surgical steel and plastic.

5. The device of claim 1, wherein the flexible shaft comprises a coil pipe.

6. The device of claim 1, wherein the flexible shaft comprises a cylinder defining a plurality of cut-out features.

7. The device of claim 1, wherein the translating member is a wire.

8. The device of claim 7, wherein the wire is a tri-layer steel wire.

9. The device of claim 1, wherein the end effector comprises:
a shuttle coupled to the translating member, wherein the shuttle is configured to move distally and proximally, wherein the shuttle comprises a first pin extending away from a longitudinal axis of the end effector and a second pin opposite the first pin;
wherein the first jaw member comprises a proximal cam member defining a first slot for receiving the first pin; and
wherein the second jaw member comprises a second proximal cam member defining a second slot for receiving the second pin.

10. The device of claim 9, wherein the first and second slots are positioned such that proximal motion of the shuttle forces the end effector into the closed position and distal motion of the shuttle forces the end effector into the open position.

11. The device of claim 1, wherein the end effector comprises:
a shuttle configured to move distally and proximally in response to motion of the actuator;
a first link having a proximal end coupled to a distal portion of the shuttle and a distal end coupled to the first jaw member;
a second link having a proximal end coupled to a distal portion of the shuttle and a distal end coupled to the second jaw member;
wherein the first and second links are coupled to the first and second jaw members at a position such that proximal motion of the shuttle forces the end effector into the open position and distal motion of the shuttle forces the end effector into the closed position.

12. The device of claim 1, further comprising:
an outer coupler connected to a distal portion of the flexible shaft; and
an inner coupler connected to the outer coupler, the translating member, and the end effector, wherein the inner coupler is rotatable relative to the outer coupler.

13. The device of claim 12, wherein rotation of the translating member causes the inner coupler and the end effector to rotate relative to the flexible shaft.

14. The device of claim 1, wherein the first jaw member comprises a first electrode positioned at a distal portion of the first surface.

15. The device of claim 14, wherein the first jaw member further comprises an electrically insulating material positioned to isolate the first electrode from a remainder of the first jaw member.

16. The device of claim 1, wherein a distal portion of the first jaw member defines a first electrode.

17. The device of claim 16, wherein the first electrode is substantially rounded.

18. The device of claim 16, wherein the first electrode is in the shape of a proximally pointing hook.

19. The device of claim 16, wherein the first electrode is substantially positioned on a surface opposite the first surface.

20. The device of claim 16, further comprising an electrically insulating material positioned between the first electrode and a remainder of the first jaw member.

21. The device of claim 1, wherein the first jaw member defines a hollow lumen and wherein the device further comprises a first electrode extending distally through the hollow lumen, wherein the first electrode defines a wire portion extending through the lumen and an active portion extending beyond the first jaw member.

22. The device of claim 21, wherein the first electrode is translatable distally and proximally.

23. The device of claim 21, wherein the active portion of the first electrode defines a proximally pointing hook.

24. A curved-jaw dissector device for use in endoscopic surgical procedures, the device comprising:
an end effector comprising:
a first jaw member defining a first surface; and
a second jaw member defining a second surface, wherein the first and second jaw members have a common pivot point such that the end effector has an open position where the first and second surfaces are pivoted away from one another and a closed position where the first and second surfaces are pivoted towards are towards each other, and wherein the first jaw member and the second jaw member are curved away from a longitudinal axis of the end effector;
a shuttle positioned substantially on a longitudinal axis of the end effector and configured to move distally and proximally;
a first link having a proximal end coupled to a distal portion of the shuttle and a distal end coupled to the first jaw member; and
a second link having a proximal end coupled to a distal portion of the shuttle and a distal end coupled to the second jaw member, wherein the first and second links are coupled to the first and second jaw members at a position such that proximal motion of the shuttle forces the end effector into the open position and distal motion of the shuttle forces the end effector into the closed position;
a flexible shaft extending proximally from the end effector;
a handle coupled to a proximal portion of the flexible shaft;
a translating member extending from the handle, through the flexible shaft, to the shuttle, wherein the translating member is coupled to the handle at an actuator having a first and a second position such that placing the actuator in the first position causes the translating member to move the shuttle proximally and placing the actuator in the second position causes the translating member to move the shuttle distally.

25. The device of claim 24, wherein the first and second jaw members comprise a material selected from the group consisting of surgical steel and plastic.

26. The device of claim 24, wherein the flexible shaft comprises a coil pipe.

27. The device of claim 24, wherein the flexible shaft comprises a cylinder defining a plurality of cut-out features.

28. The device of claim 24, wherein the translating member is a wire.

29. The device of claim 28, wherein the wire is a tri-layer steel wire.

30. The device of claim 24, further comprising:
an outer coupler connected to a distal portion of the flexible shaft; and
an inner coupler connected to the outer coupler, the translating member, and the end effector, wherein the inner coupler is rotatable relative to the outer coupler.

31. The device of claim 30, wherein rotation of the translating member causes the inner coupler and the end effector to rotate relative to the flexible shaft.

32. The device of claim 24, wherein the first jaw member comprises a first electrode positioned at a distal portion of the first surface.

33. The device of claim 32, wherein the first jaw member further comprises an electrically insulating material positioned to isolate the first electrode from a remainder of the first jaw member.

34. The device of claim 24, wherein a distal portion of the first jaw member defines a first electrode.

35. The device of claim 34, wherein the first electrode is substantially rounded.

36. The device of claim 34, wherein the first electrode is in the shape of a proximally pointing hook.

37. The device of claim 34, wherein the first electrode is substantially positioned on a surface opposite the first surface.

38. The device of claim 34, further comprising an electrically insulating material positioned between the first electrode and a remainder of the first jaw member.

39. The device of claim 24, wherein the first jaw member defines a hollow lumen and wherein the device further comprises a first electrode extending distally through the hollow lumen, wherein the first electrode defines a wire portion extending through the lumen and an active portion extending beyond the first jaw member.

40. The device of claim 39, wherein the first electrode is translatable distally and proximally.

41. The device of claim 39, wherein the active portion of the first electrode defines a proximally pointing hook.

42. A curved-jaw dissector device for use in endoscopic surgical procedures, the device comprising:
an end effector comprising:
a first jaw member defining a first surface; and
a second jaw member defining a second surface, wherein the first and second jaw members have a common pivot point such that the end effector has an open position where the first and second surfaces are pivoted away from one another and a closed position where the first and second surfaces are pivoted towards are towards each other, and wherein the first jaw member and the second jaw member are curved away from a longitudinal axis of the end effector;
a flexible shaft extending proximally from the end effector;
a handle coupled to a proximal portion of the flexible shaft, wherein the handle comprises an actuator having a first position and a second position;
a translating member extending from the handle, through the flexible shaft, to the end effector, wherein the translating member is coupled to the handle at an actuator having a first and a second position such that placing the actuator in the first position causes the end effector to be in the closed position and placing the actuator in the second position causes the end effector to be in the open position;
wherein the handle further comprises a sleeve extending proximally from a distal portion of the handle to the actuator.

43. The device of claim 42, wherein the first jaw member comprises:
a first wing feature extending away from a longitudinal axis of the first jaw member, wherein a narrow end of the first wing feature is pointed distally; and
a second wing feature extending away from the longitudinal axis of the first jaw member and opposite the first wing feature, wherein a narrow end of the second wing feature is pointed distally, and wherein leading edges of the first and second wing features are sharpened.

44. The device of claim 42, wherein the first and second jaw members comprise a material selected from the group consisting of surgical steel and plastic.

45. The device of claim 42, wherein the first and second wing features comprise a material selected from the group consisting of surgical steel and plastic.

46. The device of claim 42, wherein the flexible shaft comprises a coil pipe.

47. The device of claim 42, wherein the flexible shaft comprises a cylinder defining a plurality of cut-out features.

48. The device of claim 42, wherein the translating member is a wire.

49. The device of claim 48, wherein the wire is a tri-layer steel wire.

50. The device of claim 42, wherein the end effector comprises:
a shuttle coupled to the translating member, wherein the shuttle is configured to move distally and proximally, wherein the shuttle comprises a first pin extending away from a longitudinal axis of the end effector and a second pin opposite the first pin;
wherein the first jaw member comprises a proximal cam member defining a first slot for receiving the first pin; and
wherein the second jaw member comprises a second proximal cam member defining a second slot for receiving the second pin.

51. The device of claim 50, wherein the first and second slots are positioned such that proximal motion of the shuttle forces the end effector into the closed position and distal motion of the shuttle forces the end effector into the open position.

52. The device of claim 42, wherein the end effector comprises:
a shuttle configured to move distally and proximally in response to motion of the actuator;
a first link having a proximal end coupled to a distal portion of the shuttle and a distal end coupled to the first jaw member;
a second link having a proximal end coupled to a distal portion of the shuttle and a distal end coupled to the second jaw member;
wherein the first and second links are coupled to the first and second jaw members at a position such that proximal motion of the shuttle forces the end effector into the open position and distal motion of the shuttle forces the end effector into the closed position.

53. The device of claim 42, further comprising:
an outer coupler connected to a distal portion of the flexible shaft; and
an inner coupler connected to the outer coupler, the translating member, and the end effector, wherein the inner coupler is rotatable relative to the outer coupler.

54. The device of claim 53, wherein rotation of the translating member causes the inner coupler and the end effector to rotate relative to the flexible shaft.

55. The device of claim 42, wherein the first jaw member comprises a first electrode positioned at a distal portion of the first surface.

56. The device of claim 55, wherein the first jaw member further comprises an electrically insulating material positioned to isolate the first electrode from a remainder of the first jaw member.

57. The device of claim 42, wherein a distal portion of the first jaw member defines a first electrode.

58. The device of claim 57, wherein the first electrode is substantially rounded.

59. The device of claim 57, wherein the first electrode is in the shape of a proximally pointing hook.

60. The device of claim 57, wherein the first electrode is substantially positioned on a surface opposite the first surface.

61. The device of claim 57, further comprising an electrically insulating material positioned between the first electrode and a remainder of the first jaw member.

62. The device of claim 42, wherein the first jaw member defines a hollow lumen and wherein the device further comprises a first electrode extending distally through the hollow lumen, wherein the first electrode defines a wire portion extending through the lumen and an active portion extending beyond the first jaw member.

63. The device of claim 62, wherein the first electrode is translatable distally and proximally.

64. The device of claim 62, wherein the active portion of the first electrode defines a proximally pointing hook.
